(19) United States
(12) Patent Application Publication
(10) Pub. No.: US 2005/0100959 A1
(43) Pub. Date: May 12, 2005

(54) Device and method for detecting the presence of an analyte

(76) Inventors: Liberty Sinbanda, Harare (ZW); Sarah Maria Diana Guido De Saeger, Aalst (BE); Carlos Van Peteghem, De Pinte (BE)

Correspondence Address: William M Lee Jr;Barnes & Thornburg, P O Box 2786, Chicago, IL, 60690-2786, US

(21) Appl. No.: 10/467,562

(22) PCT Filed: Feb. 13, 2002
(86) PCT No.: PCT/EP02/01496

(30) Foreign Application Priority Data
Feb. 13, 2001 (EP) 01870025.2

(51) Int. Cl. G01N 33/53; C12M 1/34

(52) U.S. Cl. 435/7.1; 435/287.2

(57) ABSTRACT

The invention relates to a device for detecting the presence of an analyte in an interfering fraction containing fluid or semi-fluid sample, said device comprises:(a) a transparent housing,(b) inlet means for the sample to be analyzed,(c) outlet means, and (d) at least two discrete superposed layers being a first and a second layer through which at least a part of the sample is able to be transported in said order, characterized in that the first layer comprises an adsorbent medium capable of adsorbing at least a part of the interfering fraction of the sample and the second layer comprises an adsorbent medium containing an analyte-receptor capable of retaining the analyte. The invention further relates to the use of said device for detecting the presence of an analyte in an interfering fraction containing a fluid or semi-fluid sample under investigation and to a method for detecting the presence of an analyte in an interfering fraction containing a fluid or semi-fluid sample.

### FIELD OF THE INVENTION

The present invention is related to a device and method for detecting the presence of an analyte. It relates in particular to a chromatography screening procedure for assessing toxins, contaminants and clinical compounds frequently encountered in water, food, feed and body fluid samples. More particularly, in the present invention the solid-phase clean-up step and detection of an analyte of interest, e.g. a toxin or contaminant, are carried out simultaneously in one single device.

### BACKGROUND OF THE INVENTION

Our modern environment contains a lot of different substances and some of them are toxic. Type of toxins and other contaminants encountered in the environment are for instance bacterial toxins, mycotoxins, plant toxins, pesticides, hormones and antibiotics. Some toxins and contaminants are very stable and produce severe illness when ingested, inhaled, or Introduced into the body by any other means. For instance, mycotoxins are known to be poisonous, mutagenic, teratogenic or carcinogenic when consumed by humans or animals. Mycotoxins are secondary metabolites of low molecular weight produced by molds and fungi during their growth on food and feed. Mycotoxins may remain in food and feed long after the mold or fungus that produced them has died. Therefore products that are not visibly moldy or do not test positive for mold count can still contain potentially dangerous levels of mycotoxins. Diseases caused by mycotoxins in humans and animals are called mycotoxicosis and are specific to the mold species and the toxin produced. Several types of mycotoxins exist, such as aflatoxins, ochratoxins, vomitoxins, fumonisins, T-2 toxin, patulin, zearalenone . . .

Several countries have currently established or proposed regulations for control of mycotoxins (primarily the aflatoxins) in food and animal feed. In order to harmonize these regulations, the Food and Drug Administration has established guidelines for the levels of aflatoxin permitted in commodities for further processing. The permitted levels vary depending upon the intended end usage of the commodity. For instance, corn containing in excess of 20 ppb aflatoxins destined for food use by humans, for feed use by immature animals or dairy animals is rejected. Corn containing in excess of 100 ppb aflatoxins is destined for breeding cattle, breeding swine or poultry. There are also some countries with regulations for ochratoxin A (OA), trichothecenes, zearalenone, patulin and fumonisins. Maximum tolerated levels for OA range from 1 to 50 μg/kg for food and from 100 to 1000 μg/kg for animal feed. The European Commission, in an effort to standardize mycotoxin regulations between member states, has proposed a maximum permitted level of 5 μg/kg for OA in cereals.

It is obvious that the enforcement of these regulations require accurate monitoring of suspected commodities. Therefore, there is a continuous need for a very simple, rapid and inexpensive method for detecting mycotoxins.

The same applies for hormones, pesticides and antibiotics, which are often encountered in our food supply. For instance, in many situations it is of vital importance to be able to detect the presence of small amounts of antibiotics. This is the case in food industries where the increased use of antibiotics and chemotherapeutic substances in the treatment of animals has created a need for a simple, reliable and sensitive method of determination.

Many analytical methods exist in prior art for toxins, mycotoxins and other contaminants in food and feed. In general, most methods used are related to the separation and detection of analytes in a test sample using a two-steps procedure. In a first step the test sample is cleaned-up and followed by a second suitable detection step.

To date, solid phase clean-up systems are used for isolating the molecule of interest by allowing it to bind to the bonded stationary phase. Next, the unbound compounds are washed away and out of the column. The compound of interest is eluted using an appropriate buffer capable of dislodging the adsorbed molecule from the stationary phase. The eluate is evaporated to dryness and the residue re-dissolved in a smaller volume to pre-concentrate it in order to carry-out analyses such as enzyme-linked immunosorbent assay (ELISA), radio immunoassay (RIA), high performance liquid chromatography (HPLC), liquid chromatography mass spectrometry (LC-MS) and gas chromatography mass spectrometry (GC-MS). Several prior art patent and patent applications are concerned with said methods.

WO 89/03037 and U.S. Pat. No. 5,178,832 relate to a method and testing column for the selective immobilization and detection of mycotoxins in solution. It has been discovered that certain minerals, particularly various naturally occurring forms of Aluminum oxide, will preferentially bind selective mycotoxins from a mixture of mycotoxins. These adsorbents, when used in various combinations and/or in conjunction with the adsorbents of the prior art, permit the construction of detector tubes which can resolve mycotoxins in solutions and provide a semi-quantitative fluorescent determination of their concentration in feed or foodstuff samples. The detector tubes comprises transparent tubes packed with isolated layers of selected minerals. A solvent extract from a sample potentially contaminated with mycotoxins is passed through the column. As the mycotoxin mixture passes through the detector tube and is contacted by the various mineral adsorbants, selected mycotoxins are immobilized on a specific mineral while other mycotoxins and co-extracted organic compounds pass through that layer to be immobilized on subsequent downstream mineral layers. The presence of mycotoxins is determined by examining the developed detector tube under a long wave uv light source. The examples described in this patent application relate to the detection of aflatoxins and ochratoxin A (OA) in grain samples. The mycotoxins are first extracted using a suitable extraction procedure, followed by applying the sample onto the detector tube as described above.

U.S. Pat. No. 5,110,558 relates to a method and apparatus for adsorption and detection of analytes. The method and apparatus can be employed in the field for rapid adsorption of analytes and is particularly useful for detection of mycotoxins. A sample to be analyzed is prepared in solution and placed in a test tube. A tube-like adsorption column having a seal and a valve member is forcefully fed into the test tube to force solutions through the valve member into the column and through a filter and adsorbent to trap interferences. The semi-purified solution may then be analyzed for the presence of analytes. The column with the purified solution may be further employed with a second smaller adsorption column similarly equipped with a seal and valve member fitting within the first column. In similar fashion the second column may be forced into the first column to expel the solution therein into the second column and through one or more selective adsorbents for different analytes such as one or more mycotoxins. Detection of the adsorbed analyte may be made by shining a fluorescent or “black” light on the adsorbent which fluoresces to indicate presence of the analyte.

However, all these prior art analytical methods have several disadvantages. Most prior art methods are time consuming and expensive. This applies in particular for chromatographic procedures. It takes several hours to several days to complete a chromatographic analysis. In addition, extensive clean-up is often required before a sample can be applied, for example, on a HPLC column. Moreover, these techniques are not well suited for performing analyses in the field or away from a laboratory in as much as they require complex instruments and a relatively high degree of skill on the part of the person performing the analysis.

Many ELISA screening kits have also been introduced in recent years. However, sophisticated equipment and qualified personnel are still needed to perform ELISA's, and their application is restricted to laboratories.

WO99/676447 describes a multi-layer testing column comprising a plurality of membrane layers vertically stacked within the chamber of said column and include at least a plurality of solid-phase substrates each carrying a different anti-analyte. Some of the uppermost and lowermost layers are preferably filter layers, which substantially prevent passage of large particles, e.g. blood cells to other membrane layers. A sample can be placed in the chamber such that specific analytes of the sample are bound to the anti-analytes. A sensor can be located within the housing to receive a signal from the substrates and to generate a corresponding electric signal.

However, a need exists for rapid and convenient tests for analyte detection. In particular, such assays need to be simple and easy to use when performed in the field and interpreted by non-technical users. For instance, mycotoxin production occurs mostly during the harvest period after cereals, oilseeds or nuts have begun to dry, before they attain the moisture level best suited for storage. Storage of the foodstuffs under proper temperature and humidity conditions will prevent further contamination. Thus, it is important that contaminated lots are detected as early, in the food processing chain, as possible.

Therefore, the principal object of the present invention Is to provide a binding device and assay method for detecting analytes contamination for use in the field. Moreover, said device should be easy to handle, inexpensive, provide rapid and reliable results, and adaptable for field testing.

### SUMMARY OF THE INVENTION

In the present invention, a device and method is disclosed for detecting the presence or absence of an analyte in fluid or semi-fluid sample containing an interfering fraction. According to a first embodiment, the device of the invention comprises:
(a) a transparent housing,
(b) inlet means for the sample to be analyzed,
(c) outlet means, and
(d) at least two discrete superposed layers being a first cleaning-up layer and a second detection layer through which at least a part of the sample is able to be transported in said order, characterized in that the first layer comprises an adsorbent medium capable of actively adsorbing at least a part of the interfering fraction of the sample and the second layer comprises an adsorbent medium containing an analyte-receptor capable of retaining the analyte.

According to a further embodiment, the analyte-receptor present on the adsorbent medium of the second layer might be an antibody specifically recognizing the analyte of interest in the sample under investigation.

The above-disclosed device has the unique feature of the ability to trap interferences and detect analytes in one single step. Said analytes are, for example, toxins, mycotoxins, pesticides, drugs, antibiotics or hormones present in water, food, feed or body fluid samples. The adsorbent medium of the first and/or second layer is selected from the group consisting of agarose, silica, sepharose, dextrans or derivatized versions thereof. The adsorbent medium is further characterized in that at least part of said adsorbent medium comprises a derivatized surface.

According to a further embodiment the housing of the device of the invention is tubular. Furthermore, the inlet means of the device of the invention may be connectable to pressure means, for instance a hand-held portable pressure means to keep with field applications. Said pressure means are capable of exerting pressure upon said sample to force the transport of the sample from the inlet means to the outlet means. For example, the housing of the device of the invention can consist of a syringe and the pressure means of a syringe plunger.

The invention further relates to a method for detecting the presence or absence of an analyte in a fluid or semi-fluid sample containing an interfering fraction, said method comprising the following steps:
(a) applying the sample in a flow-through motion onto a two layer adsorbent medium in which the first layer is capable of actively adsorbing at least a part of the interfering fraction of said sample, and the second layer is capable of specifically retaining the analyte of interest in said sample, optionally at least part of the adsorbent medium of said first layer comprising a derivatized surface,
(b) optionally washing the two layer adsorbent medium in order to remove possible color interference of the second layer,
(c) optionally applying a predetermined amount of a binder molecule onto said two layer adsorbent medium, said binder capable of interacting with non-occupied analyte-receptor of the second layer, and able to provide detection of the presence or absence of the analyte of interest in the second layer, and
(d) finally detecting the presence or absence of said analyte of interest.

The detection of the presence or absence of the analyte of interest in the second layer is done visually or by suitable detector means. It should be noted that for field testing, it is essential that the detection can be done visually and not instrumentally, because the visual detection is simple and easy to do when performed in the field and interpreted by non-technical users.

Finally, the present invention also relates to a kit consisting of at least one of the devices of the invention as described above and one or more of the following:
(a) a pretreatment solvent capable of extracting, concentrating or dissolving the analyte of interest in the sample under investigation,
(b) pressure means connectable to the inlet means of the device and capable of forcefully exerting pressure upon said sample under investigation, to force at least part of said sample from the inlet to the outlet means of said device,
(c) a washing solution capable of removing possible color interferences of the second layer,
(d) a binder capable of interacting with non-occupied analyte-receptor of the second layer, and able to provide detection of the presence or absence of said analyte of interest in the second layer,
(e) a labeled binder molecule capable of interacting with non-occupied analyte-receptor of the second layer,
(f) a labeled derivative of the analyte molecule under investigation,
(g) a washing solution capable of removing all unbound binder from the second layer, and
(h) a substrate solution capable of reacting with said binder bound onto non-occupied analyte receptor of the second layer, and capable of generating a detectable signal.

The devices and methods of the invention permit rapid screening of important analytes, such as environmental contaminants like pesticides, food toxins and mycotoxins, antibiotics, therapeutic drugs and hormones.

Among the advantages which may be realized by the use of said device and method which embody the present invention are: speed of analysis (test takes approximately 15 minutes for semi-quantitative results), ease of use (technical expertise is not required), sensitivity, economy (minimal production costs), stability (no refrigeration is required) and flexibility (the device and associated method provide a ready-to-go field test).

The embodiments set out above and other features and additional advantages of the present invention are more fully set forth in the following detailed description below and the accompanying figure and examples.

### BRIEF DESCRIPTION OF THE FIGURES

FIG. 1. Schematical cross-section view of the device of the invention.

FIG. 2. A chromatographic profile of a roasted coffee sample spiked with OA and analyzed without an aminopropyl solid-phase clean-up step. The y-axis represents responses of the recorder (in peak area units) to the fluorescence detector signal.

FIG. 3. Chromatographic analysis of an OA-spiked (10 ng/g) roasted coffee sample by HPLC after aminopropyl solid-phase clean-up. The y-axis represents recorder responses to fluorescence detector signal.

FIG. 4. The effect of methanol concentration on the retention of OA by the aminopropyl column.

### DETAILED DESCRIPTION OF THE INVENTION

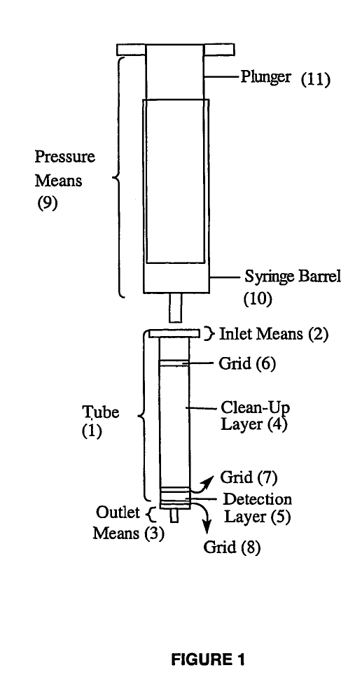

The main embodiments of the invention, and several variations of these embodiments, will be described with reference to FIG. 1. Other embodiments will be apparent to those skilled in the art.

According to a first embodiment the present invention relates to a device for detecting the presence or absence of an analyte in an interfering fraction containing fluid or semi-fluid sample, said device comprising:
(a) a transparent housing,
(b) inlet means for the sample to be analyzed,
(c) outlet means, and
(d) at least two discrete superposed layers, being a first and a second layer through which at least a part of the sample is able to be transported in said order, in which said first layer comprises an adsorbent medium comprising a derivatized surface capable of actively adsorbing at least a part of the interfering fraction of the sample and in which said second layer comprises an adsorbent medium containing an analyte-receptor, for instance an antibody capable of specifically retaining or recognizing the analyte.

Optionally, said inlet means of said device are connectable to a pressure means capable of exerting pressure upon said sample to force the transport of the sample from the inlet means to the outlet means.

In a further embodiment, in the devices of the invention, said pressure means can be a syringe plunger and said transparent housing is a syringe.

FIG. 1 depicts one of the devices for use in the present invention consisting of two superposed layers within the housing. Said device comprises a transparent housing, in particular a tube (1), inlet (2) and outlet (3) means, two superposed layers within the housing whereby the first clean-up layer (4) comprises an adsorbent material capable of adsorbing at least part of the interfering fraction of the sample, and the second detection layer (5) comprises an adsorbent material containing an analyte-receptor capable of specifically retaining the analyte. The device optionally comprises three grids, a first grid is provided above the first layer (6), a second grid in between the two layers (7) and a third grid beneath the second layer (8), providing for a physical separation barrier in-between the layers. A pressure means (9) consisting of a syringe barrel (10) operated with a plunger (11) is used to force the sample under investigation from the inlet means to the outlet means.

The invention further relates to the use of any of the devices of the Invention for detecting the presence or absence of an analyte in an interfering fraction containing fluid or semi-fluid sample under investigation, for instance said detection of the presence or absence of the analyte of interest in the second layer is done visually and is for instance based on whether a color develops or not.

A sample, containing an analyte of interest to be screened and an interfering fraction, is applied via the inlet means (2) onto said two superposed adsorbent layers. Said analyte of interest can be selected from the group consisting of toxins, mycotoxins, pesticides, drugs, antibiotics, hormones or one of their respective conjugates and derivatives. A list of possible analytes which can be screened by this invention are listed in Table 1 (not exhaustive).

As stated before, the two superposed adsorbent layers consist of a first (4) and a second (5) layer through which at least a part of the sample under investigation is able to be transported in said order. The first layer (4) comprises an adsorbent medium capable of actively adsorbing at least a part of the interfering fraction of the sample under investigation.

In order to narrow down the absorption range of interferences there has been a need to derivatize the surface of the solid support material to introduce specific chemical groups which confer a particular solid phase/matrix interference interaction. The derivatization of the solid support surface produces what is known in solid phase extraction (SPE) as a bonded matrix or bonded solid support. The solid support usually used for solid phase extraction are agarose, silica, sepharose and dextrans, including derivatized silica.

The expressions “solid phase”, “solid support phase” and “solid support material” are herein used Interchangeable and relate to the material which is used as adsorbent medium.

Thus, the expression “actively adsorbing” means that said first layer is for instance a stationary solid phase used for cleaning-up the sample, and is made-up of an adsorbent medium comprising at least partly derivatized solid support material which adsorbs targeted interferences by means of non-specific interactions (Van der Waal's), non-polar, polar or ionic interactions. One example of a derivatizing molecule is for instance carbon, to form e.g. Si—O—C₁₈Hₙ. Other examples are described below.

Bonded silica supports or bonded silica sorbents are prepared by reaction of the surface hydroxyl groups (silanols) with halo- or alkoxysilyl derivatives, resulting in the covalent bonding of a wide range of functional groups. To give the solid support, for instance the silica support the desired properties for a particular adsorption, extraction or separation, an organic moiety is attached to the solid support, e.g. the silica. The solvated bonded solid supports offer through the organic moiety an array of chemical environments which can be selected for specificities suitable for the interference. The expression “solvated” as used herein relates to a state wherein a bonded solid phase interacts with a solvent whereby the derivatizing compound on the surfaces interacts with the liquid in such a way as though it was in solution. Bonded silica solid supports exhibit unusual physical stability. They do not shrink or swell in contact with aqueous or organic solvents. The bonded silica solid phase particles are rigid and will tolerate a high viscosity flow of samples and solvents when these materials are packed into small extraction columns.

According to a further embodiment the invention relates to a device as described above wherein said solid phase adsorbs interferences in three different ways namely non-polar, polar and ionic. Non-polar interactions are those based on the dispersion forces (van der Waal's forces) that occur between the carbonaceous component of the interference and the functional group of the derivatized solid support surface. Van der Waal's forces of attraction are non-bonding interactions and are only a function of the surface area of the inter-molecular contact. The principal non-polar chemical groups used for derivatization are those with the C₁₈, C₈, C₂, cyclohexyl and phenyl groups whose long carbon chains offer a large surface area for inter-molecular interaction. For polar interactions various sorbent phases are used Including aminopropyl, cyanopropyl, diol, N-propyl-ethylene-diamine. Hydrogen bonding interactions are the polar interactions most widely used. Hydroxyl and amino groups are the common hydrogen bond donors and these typically interact with other groups containing oxygen, nitrogen and sulphur atoms. The third form of derivatization provides ionic-based interactions. This principle is based on the attraction of positively or negatively charged compounds to adsorb onto the stationary bonded phase. Bonded chemical groups for ionic interactions include diethylaminopropyl, trimethylaminopropyl, benzenesufonylpropyl, sulfonypropyl, carboxymethyl and the weekly ionic aminopropyl, and N-propylethylene-diamine (PSA). The solvated form of ionic matrices or the derivatized solid supports offers one or more charged groups (positive or negative) to which an interference with an opposite charge will bind to.

Therefore, according to a further embodiment, the invention relates to a device wherein the surface of the adsorbent solid support comprises at least one of the following functional, chemical groups: octadecyl, octyl, ethyl, cyclohexyl, phenyl, aminopropyl, cyanopropyl, diol, n-propyl-ethylene-diamine, diethylaminopropyl, trimethylaminopropyl, benzenesulfonylpropyl, sulfonylpropyl and carboxymethyl. It should be obvious to the man skilled in the art, that the modifications or functional groups which can be displayed by the adsorbent medium are not restricted to the above list, which is merely given to provide examples. For instance, as explained above, alkyl groups containing long carbon chains, such as from C₈ to C₁₈ or even longer are also envisaged as possible functional groups.

In the present invention the solid phase clean-up step is used for trapping compounds in samples which may otherwise interfere with subsequent analysis steps. For instance, the interferences may influence capturing of an analyte of interest on the second adsorbent layer of the device. Additionally, said interferences may also interfere with the subsequent detection reaction. Samples with interfering matrices may range from food, feed, industrial waste-water, urine, to blood.

The main principle is to use the stationary bonded phase to absorb and trap interferences while the analyte remains dissolved in the mobile phase and is subsequently absorbed by the second adsorbent layer of the device.

This invention has also the advantage that the reagents are compatible with both the clean-up and assay part, whereas this is not the case in conventional solid-phase extraction methods. Furthermore, to effectively increase the sensitivity of the assay, the sample is often pretreated by dissolving or extracting it with a specific solvent prior to applying said sample onto the two layer adsorbent medium. Said pretreatment may extract, concentrate or dissolve the analyte from the sample. For instance, a diluent is used which creates an environment most favorable to the analyte. This decreases the solid/mobile phase partition co-efficient in favor of the mobile phase. The analyte is then directly eluted as the sample is applied through the first adsorbent medium of the device. The interfering sample matrices are retained on the first solid phase by the specific modes of interactions provided for by the chemical environment. The second layer (5) of device of the invention comprises an adsorbent medium containing an analyte-receptor capable of retaining the analyte of interest. The adsorbent medium is a solid support material onto which an analyte-receptor is present. The solid support of the second layer should be that which supports immunological reactions. For instance, agarose, sepharose and dextrans are solid supports used in immunological and immunoaffinity solid phases. The solid support material can be an actively derivatized matrix consisting of one of the solid support media as described above and used for the first layer of the device. The analyte-receptor refers to a molecule which actively reacts with derivatized solid support for instance CNBr-activated Sepharose capable of binding specifically with the analyte.

Sepharose is bead-formed agarose gel which displays all the features required for a successful immobilization of biologically active molecules. The hydroxyl groups on the sugar residues are easily derivatized for covalent attachment of a ligand. The open-pore structure and the exclusion limit of Sepharose 4B in gel filtration (MW 20×106) makes the interior of the matrix available for ligand (e.g. analyte receptor) attachment and ensures good binding capacities. Sepharose 4B exhibits extremely low non-specific adsorption.

Adsorbents based on Sepharose are stable under a wide range of experimental conditions such as high and low pH, detergents and dissociating agents.

CNBr-activated Sepharose 4B enables ligands containing primary amino groups to be safely, easily and rapidly immobilized by a spontaneous reaction.

The analyte-receptor may be an antibody, other protein, peptide or peptide fragment, binding moiety or other binding partner specifically recognizing the analyte.

Therefore, according to a further embodiment, the second layer of the device, to which the analyte binds uses the immunoaffinity principle based on an antibody-analyte interaction. For instance, in case the analyte is a mycotoxin, the analyte-receptor is an antibody specifically recognizing said mycotoxin. In Table 1 toxicants and other contaminants and matrices in which they occur are matched with their antibodies and companies they can be obtained from.

Said antibody refers to both monoclonal and polyclonal antibodies, capable of specifically recognizing immunologically active parts or specific epitopes of the analyte of interest. The term “specifically recognizing” implies that there is substantially no cross-reaction of the antibody with other components than the analyte. The antibodies according to the invention may be produced according to techniques which are known to those skilled in the art. Monoclonal antibodies may be prepared using conventional hybridoma technology as described by Kohler and Milstein (Kohler F. and Milstein C. Nature 256, 495; 1975). This classical method comprises producing any hybridoma formed by, on the one hand, isolating splenic lymphocytes of an animal, particularly a mouse or a rat immunized against an analyte of the present invention or a fragment as defined above, and cells of a myeloma cell line on the other hand, and selecting said hybridoma for the ability to produce the monoclonal antibodies recognizing the analyte which has been initially used for the immunization of the animals.

Alternative to preparing monoclonal antibody-secreting hybridomas, a monoclonal antibody directed against an analyte of the invention can be identified and isolated by screening a recombinant combinatorial immunoglobulin library (e.g., an antibody phage display library) with the analyte of interest.

Additionally recombinant antibodies, such as chimeric and humanized monoclonal pantibodies, comprising both human and non-human portions, which can be made using standard recombinant DNA techniques, are within the scope of the invention.

Furthermore, the invention also relates to a device wherein in the second layer a predetermined space of said second layer comprises a predetermined amount of the analyte molecule to be detected, said analyte molecule labeled with an enzyme or a bioluminescent, chemiluminescent, phosphorescent or fluorescent molecule, said enzyme preferably similar to the enzyme which is used in the assay performed. Said devices are useful in the detection of the presence or absence of an analyte in a sample, for instance because they provide for an internal standard, which may give a more quantitative estimation of the analyte present in the invention, and at the same time, may serve as a control for the reliability of the assay.

According to one embodiment, in said device, two layers can be created within the second layer, (a) an anti-enzyme (internal control) layer and (b) an anti-analyte layer. The device thus may consist of two layers (1) a solid phase clean-up and (2) immunological layer consisting of (a) an anti-enzyme layer and (b) an anti-analyte layer.

The immunoaffinity principle is a basic principle for instance used in immunoaffinity columns (IAC). These columns contain a bed of a solid support material to which anti-analyte antibodies are covalently bonded. A sample containing the analyte is applied onto the column and the antibodies specifically bind the analyte after which all unbound materials are washed off and the analyte is finally eluted separately. The eluate is taken for analysis by either ELISA, RIA, HPLC, LC-MS or GC-MS. Enzyme-linked immunosorbent assays (ELISA) are also based on antibody-analyte interactions and can be used for both qualitative and quantitative analyses. These are formated as microtitre plates, the assay and results of which are carried out and interpreted in a laboratory environment using a microtitre plate reader. Other antibody-antigen systems include immunochromatographic systems comprising of a membrane along which the analyte diffuses until it reaches a site on the membrane where the anibody is bound. In another ELISA format achronymed enzyme-linked immunofiltration assay (ELIFA) or flow-through immunoassay the sample is applied directly onto a membrane where the antibody is spotted while an absorbent material draws the samples through the membrane bringing the analyte to the antibody sites. These assays can be carried out in both the lab and field and results interpreted visually. Radio immunoassays (RIA) also utilize the antibody-antigen reaction system, and are highly sensitive. However, their detection system utilizes radioactive decay which may result in handling problems. RIA applications are mostly in cell biology, for example, signal transduction and cytoplasm-based assays for the analyte detection and quantification.

The invention not only relates to devices but also to methods for detecting the presence or absence of an analyte in an interfering fraction containing fluid or semi-fluid sample, said method comprising the following steps:
(a) applying the sample in a flow-through motion onto a two layer adsorbent medium in which the first layer is capable of actively adsorbing at least a part of the interfering fraction of said sample, and the second layer is capable of specifically retaining the analyte of interest in said sample, further characterized in that at least part of said adsorbent medium of the first layer comprises a derivatized surface,
(b) optionally washing the two layer adsorbent medium in order to remove possible color interference of the second layer,
(c) optionally applying a predetermined amount of a binder molecule onto said two layer adsorbent medium, said binder molecule capable of interacting with non-occupied analyte-receptor of the second layer, and optionally washing unbound binder molecule,
(d) finally detecting the presence or absence of said analyte of interest.

In a further embodiment, in step (a) of the above method, the second layer is capable of specifically retaining the analyte of the sample for instance by the presence in said layer of an antibody, specifically recognizing the analyte under investigation.

However, the earlier described enzyme-based immunological assays of the prior art clearly differ from the invention. In said assays an extraction and clean-up step precedes and is performed separately from the immunological assay itself. On the contrary, in the present invention the solid phase clean-up step and immunoassaying of the sample are carried out simultaneously in a single assay and in one single device containing the two adsorbent layers of the invention as described above. The analyte is largely prevented from binding to the bonded stationary phase of the first layer and is therefore loaded directly onto the second layer, while interferences stay bound on the solid phase part of the first layer.

The analyte bound onto the second layer can be detected directly, for instance aflatoxins emit fluorescent light under longwave UV.

An interesting embodiment of the invention, however, is a method in which the presence of the analyte of interest in the second layer is done visually, for instance based on whether a color develops or not. Visual detection is essential for field testing and provides for quantitative or at least semi-quantitative results, for instance by detection or visual interpretation of different intensities of the color, for instance the blue color.

It is further described in separate embodiments how the above more general method is used in combination with additional steps and reagents to obtain a variety of possible methods or assays.

For instance, alternatives of the method described above, are methods wherein step (b) is present, or step (c) is present, or wherein both step (b) and (c) are present.

Alternatively, in the methods of the invention, step (c) is replaced by step (c′), wherein in step (c′) a predetermined amount of a binder molecule, labeled with an enzyme or a bioluminescent, chemiluminescent, phosphorescent or fluorescent molecule, is applied onto said two layer adsorbent medium, said binder molecule capable of interacting with non-occupied analyte-receptor of the second layer. Said binder is able to provide detection of the absence or presence of the analyte of interest in the second layer. Therefore, a suitable label is attached or conjugated to the binder, said label being detected and/or quantified. Examples of suitable labels include enzymes capable of reacting to produce a colored reaction product, such as horseradish peroxidase and alkaline phosphatase. Molecules capable of producing detectable light are also envisaged as labels, for instance molecules such as μμbioluminescence, chemiluminescence, phosphorescence and fluorescence, and particles such as carbon black, colored latex beads or gold particles.

Furthermore, the methods of the invention may comprise a step wherein the sample under investigation is pre-treated by dissolving or extracting it with a specific solvent prior to applying said sample onto the two layer adsorbent medium. In particular, said pretreatment extracts, concentrates or dissolves the analyte from the sample.

Optionally, the two layer adsorbent medium can also be washed in order to remove all ossible color interference of the second layer. Said washing can be done using a conventional buffer, for example phosphate buffered saline, Tris buffered saline or water.

The binder can be detected directly, or alternatively after the addition of a substrate. In case a substrate is used, the two adsorbent layers of the device will first be washed in order to remove all unbound binder from the second layer. Said washing can be done using a conventional buffer as described above. Said substrate solution can be, for instance, a chromogen such as Color Burst®, p-Nitrophenyl Phosphate (pNPP), 5-Bromo-4-Chloro-3-Indolyl Phosphate/Nitro Blue Tetrazolium (BCIP/NBT), Fast Red/Naphthol AS-TR Phosphate, 2,2′-Azino-bis(3-Ethylbenzthiazoline-6-Sulfonic Acid) (ABTS), o-Phenylenediamine (OPD), 3,3′,5,5′-Tetramethylbenzinedine (TMB), 5-Aminosalicylic Acid (5AS), 3,3′-Diaminobenzidine Tetrahydrochloride (DAB), D(−)-Luciferin (for Bioluminescence), POD.

Said substrate is capable of reacting with the binder bound onto the non-occupied analyte-receptor of the second layer and capable of generating a detectable signal. In the bsence of any analyte in a sample under investigation, all the binder will be trapped in the second layer/detection zone yielding a high signal. The presence of analyte in the sample produces a decrease in signal proportionately as the amount of analyte in the sample increases. The intensity of the signal developed can be compared to that of known quantities of analytes applied to similar devices in the same manner and thus representing “reference” devices, or can be applied to a device including an internal standard as described earlier.

For instance, the substrate can consist of a chromogen which is converted to a blue color by an enzyme conjugated to the binder. In this case, the interpretation of the result can be done visually and is based on whether a blue color develops or not. When the sample contains a particular amount of an analyte or more no color develops. When the analyte concentration is lower than this critical concentration level a blue color develops.

According to a further embodiment, additional steps are present in between steps (c) or (c′) and (d) of the method of the invention as described above consisting of
optionally washing said two layer adsorbent medium in order to remove all unbound binder from the second layer, and
applying a substrate solution onto said two layer adsorbent medium, said substrate solution capable of reacting with the binder bound onto the non-occupied analyte-receptor of the second layer and capable of generating a detectable signal.

According to a further alternative embodiment, the invention relates to any of the methods as described above wherein step (c) or (c′) is replaced by step (c″) wherein in step (c″) as one example of a binder molecule, a predetermined amount of the analyte molecule is applied onto said two layer adsorbent medium to be detected, said analyte molecule labeled with an enzyme or a bioluminescent, chemiluminescent, phosphorescent or fluorescent molecule, and furthermore, said labeled analyte molecule capable of interacting with non-occupied analyte-receptor of the second layer. The addition of a labeled analyte molecule at that moment in the assay method, provides a means for detection of the absence or presence of the analyte of interest retained from the sample by the analyte-receptor in the second layer.

Also this method may contain the additional steps between step (c″) and (d) of
optionally washing said two layer adsorbent medium in order to remove all unbound labeled analyte molecule from the second layer, and
applying a substrate onto said two layer adsorbent medium, said substrate capable of reacting with the labeled analyte molecule bound onto the non-occupied analyte-receptor of the second layer and capable of generating a detectable signal.

Thus, detecting the presence or absence of the analyte of interest in the second layer can be done by the naked eye. Alternatively, a suitable detector means can be used, capable of electronically detecting the color developed and providing a more exact quantification. Such a quantification would allow the calculation of the level of analyte in the test sample. Any detection method may be assisted by computer technology and detection methods can therefore be automated by various means. A suitable detector might be, for instance, a colorimeter.

According to another embodiment, the inlet means of the device of the invention are connectable to pressure means (9) capable of exerting pressure upon the sample to force the transport of the sample from inlet means (2) to outlet means (3). On FIG. 1, a pressure means (9) suitable for use in the invention is depicted. In this case, the pressure means consists of a syringe barrel (10), and the sample is applied onto the device (for instance, a tube) by means of a syringe plunger (11). Alternatively, in case the housing of the device is a e itself, the pressure means can consist of a syringe plunger which fits into said syringe.

This invention thus employs frontal elution or elution chromatography. As pressure is continuously applied on e.g. the plunger, the mobile phase carries the dissolved analyte towards the outlet means (3), for instance the end of the tube in FIG. 1. As a result the analyte of interest is quickly loaded onto the second adsorbent layer of the device where it will selectively bind to. In this chromatographic elution system the breakthrough volume is significantly reduced. The breakthrough volume is defined as the sample volume eluted from the outlet means until analyte concentration reaches 1% of the analyte concentration added at the inlet means. The breakthrough volume corresponds to the largest sample volume that can be processed without significant loss of analyte and for which recovery after elution for sample volumes less than the breakthrough volume will be 100% in the absence of irreversible sorbent interactions. It is the breakthrough volume that is most important in determining the suitability of an adsorbent medium for a particular isolation procedure.

The device and method of the present invention have several advantages. These advantages mainly result from the fact that the present invention permits a simultaneous clean-up and detection of an analyte in a sample under investigation. In addition, interpretation of the results can be done visually. Furthermore, the device of the current invention is easy to fabricate using readily available, relatively inexpensive materials. Moreover, the test method which employs this device is rapid and easily performed. The reagents and equipment needed for said method are portable and stable at ambient conditions and safe to use. Yet another important advantage is that the device and method are particularly useful for field testing and screening of samples for the presence of analytes, without the need for extensive training or expensive laboratory equipment.

In summary, the screening method and device of the invention provides fast, simple, cost-effective and reliable information when operated under field conditions.

The invention can be applied as a general detection method for a large variety of target analytes. For instance, the device can be used for the detection of toxins or mycotoxins in food and feed, for pesticides in water, hormones and antibiotics in milk or body fluids. The device will prove useful as a regulatory tool to monitor mycotoxin contamination in agricultural commodities, prepared foods and mixed feeds at buying locations, field installations, processing lines, grain elevators, feed lots and the like. It can facilitate the rapid differential diagnosis of mycotoxicoses in animals (by testing body fluids or tissue extracts, particularly those of the liver and kidney) and perform presumptive field analyses for mycotoxins.

Furthermore, the invention provides an easy-to-use device in doctors offices, at clinics or at home for testing the presence of hormones or therapeutic drugs in body fluids. Additionally, clinics, emergency medical technicians and policemen require an affordable and easy to use device for quickly testing for the presence of drugs of abuse in body fluids outside of a hospital setting.

According to a general embodiment the invention thus relates to any of the methods described herein in which the analyte in said sample under investigation is a member selected from the group consisting of toxins, mycotoxins, pesticides, drugs, antibiotics, hormones, and their respective conjugates, metabolites and derivatives.

The invention is further illustrated in the enclosed example describing the use of the method and device for screening for the mycotoxin ochratoxin A (OA) in roasted coffee. However, it should be appreciated by those skilled in the art that this example is merely illustrative and a great variety of embodiments are possible which employ various combinations of adsorbents and antibodies depending on the various analytes for which analysis is desired.

The housing of the device in this particular example is a tube containing two superposed adsorbent layers. The roasted coffee samples are first extracted with an appropriate organic solvent, and next applied onto the tube. The adsorbent material of the first layer will trap all possible interferences and clean-up the sample. Next, a washing solution is applied onto said tube to remove all color of the second layer. The second layer of the device uses an immunoaffinity principle based on an antibody-analyte interaction system. An antibody specifically recognizing OA is covalently bound onto the adsorbent material of the second layer. Ochratoxin present in the sample under investigation will be retained onto said second layer. Next, an amount of a labelled OA solution is applied onto the tube and will bind non-occupied antibody sites of the second layer. The tube is again washed in order to remove unbound ochratoxin. Detection can be done by the naked eye, after applying a substrate solution onto the tube capable of reacting with labelled ochratoxin bound onto the second layer of the tube. When no analyte was present in the sample under investigation, the second layer of the tube will color. No or less color develops when the analyte concentration increases in the sample.

Notwithstanding the inventive concept of applying the clean-up method and a detection method in a single device and assay, the cleaning up method has been optimized by the present inventors and can be used seperately for instance for removing an interfering fraction from a fluid or semi-fluid sample, prior to the analysis of said sample in HPLC or in a conventional or flow-through enzyme immunoassay.

In general solid phase extraction (SPE) procedures, a sample with analyte is applied over the solid phase, the targeted analyte binds to the solid phase while the rest of the sample passes through the solid phase, the solid phase is washed with buffer to remove interfering matrices, the analyte is then eluted with an appropriate solvent, the eluate is evaporated to dryness and redissolved in a smaller volume to pre-concentrate it for analysis.

The clean-up extraction method of the present invention is only a two step procedure wherein the sample is processed in a directly opposite way with the sample extraction solution used directly as the analyte eluate by providing a conducive solvent environment for the analyte. The sample is brought to the analytical immunoaffinity layer directly at a low alcohol concentration thus does not affect the immunological reactions of the second layer. This is thus only a two step method in as far as the solid phase clean-up procedure is concerned compared to the mainstream SPE principles.

Therefore, according to another embodiment the invention relates to a solid phase cleaning up method for removing an interfering fraction from a fluid or semi-fluid sample, said method comprising applying the sample in a flow-through motion onto an adsorbent medium which is capable of actively adsorbing at least a part of the interfering fraction of said sample, characterized in that said adsorbent medium comprises a solid support material selected from the group consisting of silica derivatives and wherein the surface of said solid support material is derivatized to produce a bonded matrix, for instance a bonded matrix wherein the functional chemical groups displayed at the surface of the solid support medium are chosen from octadecyl, octyl, ethyl, cyclohexyl, phenyl, aminopropyl, cyanopropyl, diol, n-propyl-ethylene-diamine, diethylaminopropyl, benzenesulfonylpropyl, sulfonylpropyl, carboxymethyl and trimethylaminopropyl.

The invention also relates to a device operable in the above described method, for instance a device wherein said absorbent medium is a bonded silica solid phase e.g., aminopropyl solid phase.

In an interesting embodiment, said cleaning up method and/or said device are used with a sample wherein the presence or absence of ochratoxin A needs to be detected.

Aminopropyl bonded silica is prepared by the reaction of the silanols with halo- or alkoxylyl derivatives, resulting in the covalent bonding of a wide range of functional groups. The high surface coverage that can be achieved during the bonding process means that the adsorptive characteristics of the bonded silica sorbent are largely a function of the characteristics of the phase covalently bonded to the silica surface:
—Si—CH₂—CH₂—CH_{2 —NH}₂

Aminopropyl is a polar solid-phase and the polarity is due to a concentration of negative charge on one end of the molecule and a concentration of positive charge on the other end. This is brought about by having the negative electrons within the atoms of the molecules shift towards those molecules which are most capable of attracting them. This shift produces a molecular dipole. Other polar molecules will be attracted since each of them, in turn, have a positive and negative end. In aminopropyl the polar characteristic is brought about by the amino-group. Amines have at least one sp3 hybridized nitrogen bond to bond to as few as one hydrogen group. The nitrogen atom of primary amines has a lone pair of electrons that often in the presence of a more acidic substance is capable of donating the lone pair in forming a fourth bond making the nitrogen atom electron deficient giving it a net positive charge.
CH₃—NH₂+H⁺→CH₃—NH₃⁺
This sets the aminopropyl solid phase ready to receive an electron from a polar compound. It is for this reason that for instance in particular applications, such as for detecting OA, the extraction and hence the elution solution should contain a high concentration of methanol in order to directly elute the bulk of the OA in the extract at levels that are detectable by the enzyme immunoassay. At lower methanol concentrations lower amounts of methanol interact with both OA and aminopropyl solid phase to effectively dissolve it. At higher methanol concentrations it is envisaged that OA and the aminopropyl solid phase will be completely associated with methanol molecules to effect a direct elution as the interaction of OA with the solid phase will be somewhat impeded by the methanol. Methanol has a dipole moment of 1.6, polarity of 232.3 kJ.mol⁻¹ and a nucleophilic donor strength of 107.5 kJ.mol⁻¹. These are apparently higher than those for the amino-nitrogen. The association of OA with methanol in the sample is, therefore, expected to receive the minimum disruption as it passes through the column.

Although aminopropyl has a short carbon chain it is capable of limited non-polar interactions. Also according to the invention, the cleaning up method described above is used for actively removing an interfering fraction from said sample in a method for detecting the presence or absence of an analyte in a fluid or semi-fluid sample, for instance prior to the application of said sample in a flow-through enzyme immunoassay or in an HPLC analysis.

The invention, now being generally described, will be more readily understood by reference to the following examples, which are included merely for purposes of illustration of certain aspects and embodiments of the present invention and are not intended to limit the invention.

### EXAMPLES

### Example 1

### Device and Method for Detection of an Analyte in a Interfering Fraction Containing Sample

Since different types of gels offer various options for immunological detection, they make it possible to prepare a range of detection layers for use in the devices. These gels include CNBr-activated Sepharose 4B, Activated CH Sepharose 4B, NHS-activated Sepharose and EAH Sepharose 4B (for aminopropyl group attachment), ECH Sepharose 4B (for —COOH group attachment), while Epoxy-activated Sepharose 6B couples through hydroxyl, amino or thiol group, all these are for instance available from Pharmacia (Sweden).

A ligand (analyte receptor) solution with a specified concentration is coupled with a specified amount of gel by a method described by the manufacturer. The choice of analyte receptors, for instance antibodies, depends on the analyte to be detected and are commercially available from several suppliers.

Briefly, the gel is swelled by washing with 1 mM HCl (200 ml/g) over a sintered glass filter, the ligand is dissolved in an appropriate buffer, for instance 0.1 M NaHCO₃ (pH 8.3) buffer containing 0.5 M NaCl (5 mg ligand ml⁻¹ gel) and subsequently coupled with gel by shaking end-over-end for 2 hrs at RT or overnight at 4° C. The remaining active sites are blocked, for instance with either 0.2 M glycine (pH 8.0) or 1 M Ethanolamine for 16 hrs at 4° C. or 2 hrs at RT. Excess adsorbed ligand is washed away for instance with coupling buffer followed by 0.1 M acetate (pH 4) buffer containing 0.5 M NaCl followed by coupling buffer. The blocking agent is washed away using coupling buffer. The coupled gel buffer ratio is for instance1/3.

A separate amount of gel is swelled and blocked as described above. The coupled gel (for instance 1 ml in 3 ml buffer) is mixed with 4 times this volume of blocked gel. The mixture is brought to an equilibrium by shaking end-to-end for 3 minutes. For instance 150 μl of this mixture is pipetted into an empty column with the endcap and first grid in place (FIG. 1). Empty columns with grid can be obtained, for instance, from Varian, (Harbor City, USA). The second grid is introduced to compress the gel suspension to a final thickness of approximately 2 mm (FIG. 1). The solid phase material (for instance 200 mg) (clean-up layer) is introduced after the column is filled with buffer, for instance NaHCO₃ (FIG. 1). Various types of solid phases, for instance bonded Solid phases (anionic, ionic, polar and non-polar) are obtainable from Varian (Harbor City, USA) and J. T. Baker (Belgium). The third grid is introduced to superimpose over the solid phase material adequately compressed (FIG. 1). At this stage the column is ready for use.

A sample is collected and dissolved/extracted with a specific amount of solvent which in turn is compatible with the immunoaffinity section of the column. The sample is applied on the column through a syringe by means of a syringe plunger (FIG. 1) (Syringes are for instance from Becton Dickinson, (Temse, Belgium)).

First the sample encounters a specified amount of solid phase onto which sample matrices are adsorbed while the analyte is favorably dissolved in the solvent. The solvent flows through the solid phase part of the column (first clean-up layer) carrying the analyte to the immunoaffinity section (second detection layer). The compatibility of the solvent with the immuno-reactive section part of the column ensures that the antibodies are not affected and the analyte is bound. Any discoloration and matrices are washed off with a specified amount of washing buffer. A specified amount of enzyme-analyte concentration is applied onto the column. Any unbound enzyme conjugate is washed off with washing buffer. Then a volume of chromogen substrate is applied and a color develops on the immunoaffinity section of the column for samples pre-defined as negative and no color develops for positive samples. Chromogen substrates can be obtained for instance from Sigma (USA), Calbiochem (San Diego, USA), Pierce (Belgium), or other suppliers.

### EXAMPLE 2

### Development of a Solid Phase Immunoaffinity Column-Based Enzyme Immunoassay for the Detection of Ochratoxin A in Roasted Coffee.

A column and a method for a simultaneous clean-up and analysis/detection of OA was designed.

The main principle is to use the stationary bonded phase to adsorb and trap matrix interferences while the analyte remains dissolved in the mobile phase and is subsequently adsorbed by the immunoaffinity section. Therefore, to effectively increase the sensitivity of the assay the sample is diluted with a solvent which dissolves the analyte. Thus the diluent creates an environment most favorable to the analyte. This decreases the solid/mobile phase partition coefficient in favor of the mobile phase. The molecule is then directly eluted as the sample passes through the immunoaffinity section of the column. The matrix interferences are retained on the solid phase by the specific modes of interactions provided for by the chemical environment.

This method employs frontal elution or elution chromatography. As pressure is continuously applied on the plunger the mobile phase carries the dissolved analyte towards the outlet end of the column. Thus, the analyte of interest (OA) is quickly loaded onto the immunoaffinity section of the column where it is selectively bound and any remaining interfering substances are washed off. In this chromatographic elution system the breakthrough volume is significantly reduced. It is the breakthrough volume that is most important in determining the suitability of a sorbent for a particular isolation procedure. As demonstrated by the partition coefficient (Table 5) and the elimination of interfering peaks the aminopropyl solid phase material was reliably adopted for use in this column.

The second part of the column to which the analyte binds uses an immunoaffinity principle based on an antibody-analyte interaction system. In the present method the solid phase clean-up and the immunoassaying of the sample are carried out simultaneously in the same column.

A column as generally described in Example 1 was prepared and the method was optimized, for instance, to more specific requirements for detecting OA in a sample of roasted coffee. Anti-Ochratoxin A antibodies and HRP-OA conjugate were obtained from the Institute of Animal Sciences, Agricultural Biotechnology, (Gödöllö, Hungary). The anti-OA was coupled with the CNBr-activated gel (Pharmacla Biotech, Sweden) (for preparing the detection layer) and diluted with blocked gel as described in Example 1. Aminopropyl (solid phase material for preparing the clean up layer) was obtained from J. T. Baker (Belgium) while ColorBurst® Blue was obtained from ALerCHECK Inc. (USA).

Ground roasted coffee (5 mg) were extracted with 15 ml 50% Methanol/3% aqueous NaHCO₃ by shaking for 15 minutes. The sample was filtered with filter paper and 8 ml of extract were diluted to 20 ml with 3% aqueous NaHCO₃ to reduce the MeOH to 20%. The dilution was applied over the column at a rate of 1 drop per second. Subsequently, the column was washed with 10 ml of 3% aqueous NaHCO₃ and 100 μl of HRP-OA (1:200) in assay buffer (0.1% Casein PBS pH 7.4) was added. The column was washed once more with 10 ml of H₂O and a 50 μl volume of ColorBurst® Blue was drawn into the immunoaffinity section of the column by withdrawing the syringe plunger such as to create a backward flow or sucking action through the tip of the column. The chromogen substrate fills the immuno-reactive part of the column.

The method was further optimized as follows.

The effects of volume of sample extract were investigated in an assay by comparing 10 ml of PBS (pH 7.4) to 2, 4, 6 and 8 ml of roasted coffee extract.

The different volumes were applied onto the columns by a syringe (obtained from Becton Dickinson, Temse, Belgium), and the assay was performed essentially as described above. The syringe provided the pressure means as outlined in FIG. 1.

An intense blue color was observed for the assay in which 10 ml of PBS (pH 7.4) was applied and for the assay in which 2 ml of sample extract was applied. There was less color development in the assays for which 4, 6, and 8 ml were applied.

To carry out the assay with a total elimination of cross-reactivities 2 ml of sample extract was chosen as the most definitive assay volume to be assayed.

In order to yield more OA from the column a higher concentration of methanol was used so as to effect a higher frontal elution of OA onto the immunoaffinity section of the column. However, increasing the concentration of methanol in the sample extract to be assayed may affect the immunoaffinity section of the column hence the performance and success of the assay. To optimize the concentration of methanol in an assayed sample extract spiked with OA, the effect of different concentrations of methanol on the performance of the assay were evaluated. The concentrations investigated were 10, 15, 20 and 25% methanol.

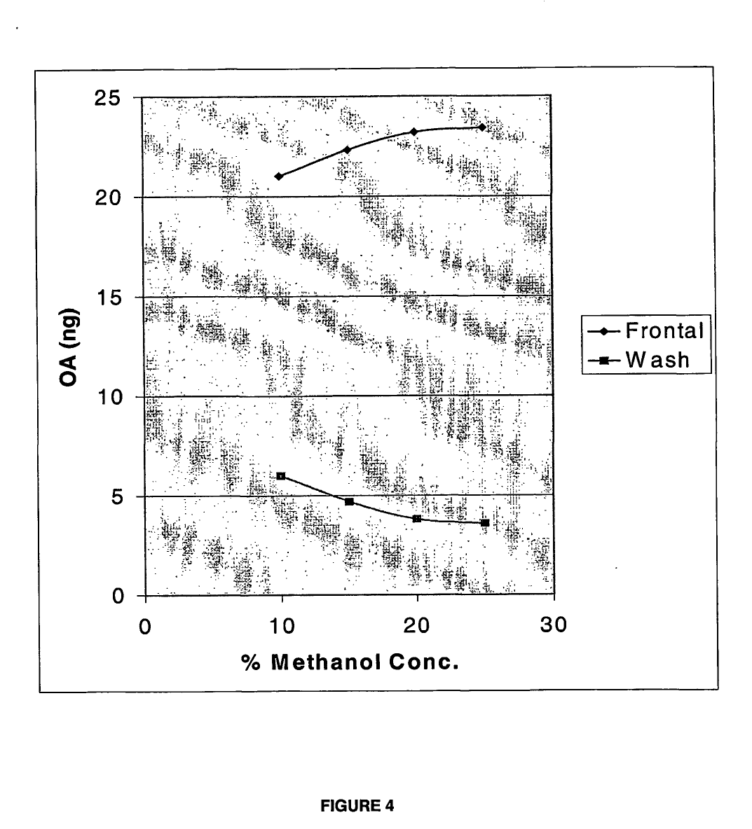

The retention capability of the aminopropyl column was greater at lower methanol concentration and tended to decrease as the methanol concentration increased from 10 to 20% (FIG. 4). The eluotropic ability of the sample extract increased with decreasing dilution factors. Though the eluotropic ability of the extract increased with increasing methanol concentration a methanol concentration of 20% was finally adopted so as to avert the negative effects of higher methanol concentrations on the immunoaffinity section of the column.

The recoveries of residual OA from the column with increasing methanol, as shown by the “wash graph” in FIG. 4, increased at a decreasing rate. Previous experience with enzyme immunoassays employing higher methanol concentrations showed no effect on the assay itself (Sibanda et al. 2000, J. Agricultural and Food Chemistry, 48: 5864-5867).

Therefore, in this case a conservative 20% methanol was chosen for use in the column-based enzyme immunoassay. The choice of a 20% methanol concentration was to ensure an efficient eluotropic effect on OA over the aminopropyl column. This thus carries a considerable quantity of OA to the immunoaffinity section of the column hence an increased assay sensitivity.

Furthermore, it was found that were no false positives recorded during the repeatability studies of the assay during 5 days (Table 6). The assay repeatability is high showing definite reliability of the assay and its applicability to routine screening of roasted coffee samples for OA.

### EXAMPLE 3

### Optimization of Solid Phase Clean-Up Method

The effect of pH and methanol on the direct elution of OA over the aminopropyl solid phase material was investigated by using three different methanol concentrations (40, 50 and 60%) for extraction and adjusting the sample extracts to different pH levels. The clean-up procedure used was as follows with different pH value. The extracts (mean pH 5.6) were adjusted to pHs 4.0, 4.5, 5.0, 5.5, 6.0, 6.5, 7.0, 7.5 and 8.0 using hydrochloric acid (HCl) and sodium hydroxide (NaOH). The sample, 3.5 ml, with the adjusted pH was passed over the aminopropyl solid phase material (200 mg) at a rate of 1 drop per second. The eluate was diluted to 60 ml with PBS (pH 7.4) and brought over an Ochratest™ IAC. OA was eluted with 4 ml of methanol. The eluate was evaporated at 40° C. under a stream of nitrogen gas and the residue was redissolved in 150 μl of methanol.

After processing, the samples were analyzed with the HPLC method.

There was no link between pH and OA recoveries over the pH ranges investigated. At 40% methanol/3% aqueous NaHCO₃ recoveries were almost nihl. However at 50% and 60% methanol recoveries increased significantly. At 50% methanol better recoveries were obtained over the pH ranges 5.0-6.0 which are relevant to a freshly extracted sample.

As it was evident, pH did not have a significant influence over the chromatographic elution of OA from aminopropyl columns. There was, therefore, no need to alter the pH of the extracts. For the detection of OA, it was however important that 50% methanol concentration be adopted as the working concentration for clean-up. The main reason being the need to dilute less to circumvent the reduction of assay sensitivities.

Further, the adsorptive and clean-up characteristics of the aminopropyl solid phase were optimized by means of establishing the most conducive chemical environment in which the interfering compounds are effectively bound. This was done by investigating the effect of increasing the NaHCO₃ concentration from 0% to 8% in the extraction solution. The extracts were cleaned-up and the eluates analyzed by HPLC to determine the dispersion of OA between the mobile and the stationary phase. This was achieved by collecting the mobile phase fraction (frontal fraction) and the stationary phase fraction (wash fraction) separately. The extraction and clean-up methods was as follows. The sample was extracted with 50 ml of methanoVo, 1.5, 3, 4, 6, and 8% aqueous NaHCO₃ (1/1, vol/vol). After filtration, 4 ml was extracted over 200 mg of an aminopropyl column at a rate of 1 drop per second into a test tube (frontal fraction). The column was washed with 2 ml of methanol/3% aqueous NaHCO₃ (1/1, vol/vol) and lastly with 1 ml absolute methanol and both washings were eluted into the same test tube. The eluate, 7 ml, was diluted to 100 ml with PBS (pH 7.4) and extracted over an Ochratest™ IAC and prepared for HPLC analysis.

Increasing the concentration of NaHCO₃ had an effect of increasing the adsorptive efficiency of the aminopropyl solid phase material. Although peaks were detected from 0% to 4% aqueous NaHCO₃ there were no peaks at 6% and 8% aqueous NaHCO₃. These results are shown in Table 3.

Since the peak disappeared completely when moving from 4% to 6% aqueous NaHCO₃, 5% aqueous NaHCO₃ was chosen as the optimum concentration and no matrix interference peak appeared afterwards.

### EXAMPLE 4

### Optimization of Solid Phase Clean-Up Prior to HPLC Analysis of Ochratoxin A in Roasted Coffee

The introduction of more sensitive High Performance Liquid Chromatography (HPLC) methods permitted the detection of trace levels of OA in roasted coffee (Terada et al. J. Assoc. Anal. Chem., 69 (1986) 960). However, the analysis of OA in coffee is still hampered by acidic substances extracted together with OA (Pittet et al. J. Agric. Food Chem. 44 (1996) 3564). The HPLC method was recently improved by the introduction of the use of immunoaffinity columns (IACs) for the clean-up of coffee products (Nakajima et al. Food Agric. Immunol. 2 (1990) 189). In a 1996 study a method was reported in which IACs were used directly after sample extraction without a clean-up step (Pittet et al. J. Agric. Food Chem. 44 (1996) 3564). Due to extensive interferences by the coffee matrix it was necessary to increase the retention time of OA to nearly 14 minutes. Later in 1997 the use of a Sep Pak Silica column for solid phase clean-up of the extract was reported and the resultant chromatograms showed a well resolved OA peak and a stable baseline (Patel et al. Food Addit. Contam. 14 (1997) 217). However, this clean-up method employed extensive washing steps using chloroform, chloroform-methanol, toluene-acetic acid and acetonitrile. In this example we describe a new clean-up method employing aminopropyl (NH₂) as the solid phase material. The method employs only three steps resulting in a sample extract which can be analyzed directly by an immunological method or further extracted by IAC for HPLC analysis.

The newly developed extraction method was used as a clean-up step prior to sample preparation for HPLC analysis. An interfering compound with a similar retention time as OA was adsorbed by the aminopropyl (NH₂) material at ≦5% NaHCO₃.

The main objective was to assess the extraction method and compare recoveries to standards representing the actual and expected quantities.

In the experimental set up, 20 g samples spiked with 0, 2.5, 5, 10, 20 and 40 ng OA.g⁻¹ were used. Samples were extracted with 50 ml of methanol/5% aqueous NaHCO₃ (1/1, vol/vol). The column was washed with 2 ml of methanol/5% aqueous NaHCO₃ (1/1, vol/vol) into the same flask and finally with 1 ml absolute methanol.

The HPLC method used was an adaptation of that described by Pittet et al. 44 (1996) 3564) The sample (50 μl) was injected manually by means of a Rheodyne manual injector (Waters, Milford, Mass., U.S.A). The HPLC system consisted of a Waters™ 600 Controller and a Waters 610 Fluid Unit (Waters, Milford, Mass., U.S.A.). The flow rate was 1 ml per min over a Supelco Discovery™ C18 (25 cm×4.6 mm, 5 μm) reversed-phase column (Supelco, Bellafonte, U.S.A.) at ambient temperature. The mobile phase used was acetonitrile/water/acetic acid (99/99/2). OA detection was achieved by means of a Waters 474 scanning fluorescence detector (Waters, Milford, Mass., U.S.A.) set at 333 nm excitation and 460 nm emission wavelengths.

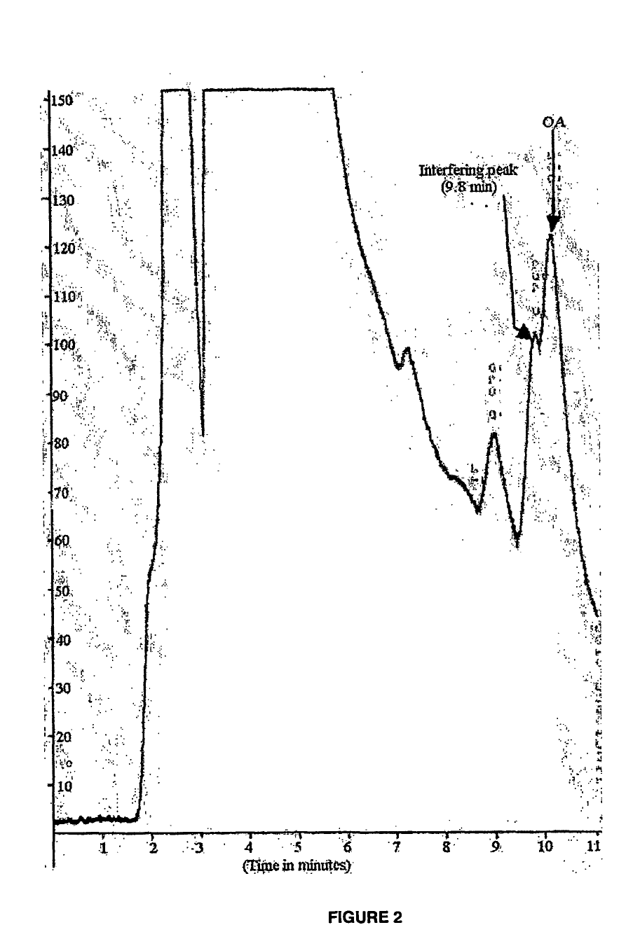

The HPLC conditions allowed retention of OA only up to 10 minutes. However, roasted coffee matrix interferences covered the chromatogram from ca. 1.5 minutes to over 15 minutes. There was a matrix peak with an identical retention time as that of OA in a blank roasted coffee sample after the IAC sample clean-up. This, therefore, masks the OA peak at ca. 10 minutes, which appears as a shoulder on matrix peaks (FIG. 2). This, therefore, illustrates the inadequacy of using IACs in isolation as a clean-up method highlighting the need to add an SPE step prior to the IAC clean-up step.

Various solid phases [trimethylaminopropyl (SAX), n-propyl-ethylene-diamine (PSA), aminopropyl, octadecyl (18), Diol (2OH) and cyanopropyl (CN] were investigated for their ability to adsorb the matrix interferences and particularly the brown coffee color and the compound interfering with the OA peak 5 (Table 2). Aminopropyl was selected for its chromatographic elution of OA and adsorption of the interfering compound. Different concentrations of NaHCO₃ were investigated and there was an observed decrease in peak area of the interfering compound with increasing NaHCO₃ concentrations (Table 3). From these results 5% aqueous NaHCO₃ was chosen as the optimum salt concentration in the extraction solution for the effective adsorption of the interfering compound on the aminopropyl solid phase material.

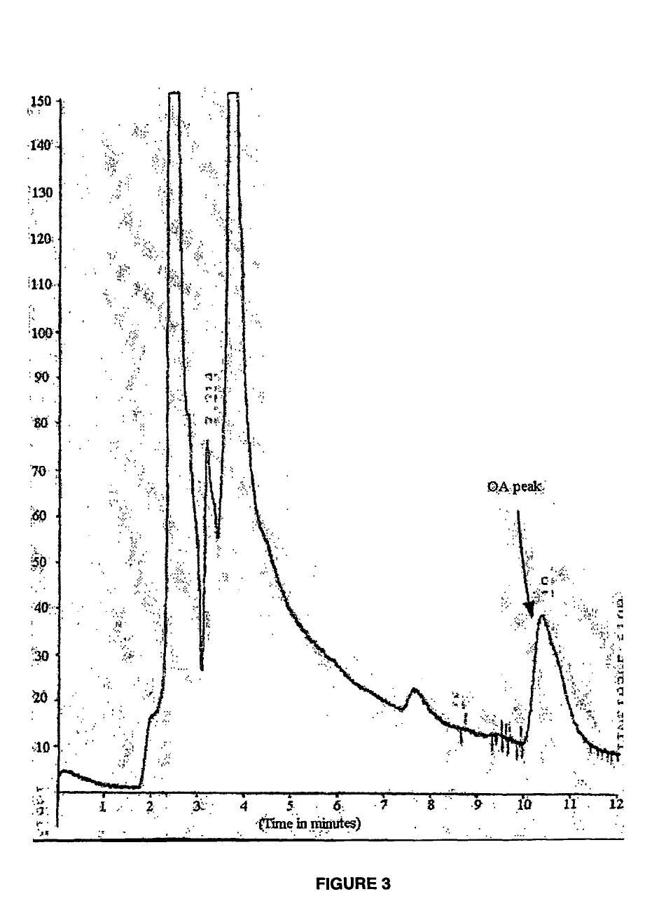

The chromatogram showed extensive elimination of matrix interferences resulting in a well resolved OA peak (FIG. 3). Method recoveries ranged from 72-84% in spiked samples (n=3 replicated twice) (Table 4). Regression (r²) of peak area on concentration for both standards and spiked samples were identical and these were 0.981 and 0.984, respectively.

The recovery of OA from the aminopropyl column followed was confirmed by derivatization to its methyl ester confirmed whereas the interfering compound peak disappeared after derivatization. The method was successfully applied to 9 commercial roasted coffee samples. The main advantages here are that the quantification of OA can be done within half the usual time it takes to analyze roasted coffee samples. The column is not overloaded and the chromatogram shows that the baseline is established first before the OA peak appears.

Recoveries were considered high enough (Table 4) for the method to be used in the analysis and confirmation of roasted coffee samples. The clean-up method employing the aminopropyl solid phase material offers an efficient system for eliminating complex matrix interferences, therefore, there is no need for an extra confirmation step as required by other published methods for the analysis of similar matrices (Tsubouchi et al. J. of Agric. Food Chem. (1998) 36: 540; Studer-Rohr et al. J. of Food and Chemic. Toxic. (1995) 33:341; Pittet et al. 1996 J. Agric. Food Chem. 44 (1996) 3564). There is also no need to switch to different mobile phases when analyzing green and roasted coffee samples. The aminopropyl column clean-up method is also compatible with the rapid field enzyme immunoassay format. No pre-conditioning of the aminopropyl column is required. The ability of the clean-up method to chromatographically elute the toxin while adsorbing matrix interferences does not require an additional methanol step. This, therefore, has an advantage of requiring lower dilution factors and method sensitivity is not affected.

TABLE 1
Toxicants, contaminants and matrices in which they occur matched with their
antibodies and companies they can be obtained from (not exhaustive).

| Contaminants/ | | | |
|---|---|---|---|
| Toxicants | Matrices | Antibodies | Companies |
| Mycotoxins | | | |
| Aflatoxin M₁, M₂, B₁, | Milk, Cheese, nuts, | Anti-AFM₁, M₂, B₁, | Sigma & ICN, IASABC |
| B₂, G₁, G₂ | Beer, cereals | B₂, G₁ and G₂ | |
| | Coffee, feed | | |
| Ochratoxin A | Beer, cereals, | Anti-OA | IASABC |
| | grape juice, wine | | |
| T-2 | Beer, cereals | Anti-T-2 | IASABC |
| Roquefortine | Cheese | Anti-roquefortine | |
| Deoxynivalenol | Cereals, beer | Anti-DON | ICN, Sigma & IASABC |
| Fumonisins | Beer, cereals | Anti-FB₁, FB₂ | ICN, Sigma, Calbiochem |
| Zearalenone | Beer, cereals, feed | Anti-Zea | ICN, Sigma & IASABC |
| Patulin | Apple juice, wine | Anti-patulin | ICN, Calbiochem |
| Hormones | | | |
| Progesterone | Milk | Anti-progesterone | Calbiochem, Sigma & ICN |
| Testosterone | Milk | Anti-testosterone | Sigma & ICN |
| Steroids | Urine | Anti-steroid | Sigma |
| β-agonists | Urine | | |
| Growth hormones | Urine, Blood | | |
| Pesticides | | | |
| Nitrophenols | Water | | |
| Organochlorine | Water | | |
| Atrazine (herbicides) | Water | Anti-atrazine | Millipore |
| Alachlor | Water | Anti-alachlor | Millipore |
| Triazines | Water | | |
| Acetamides | Water | | |
| 2,2-bis(4- | Urine | | |
| chlorophenyl) acetic | | | |
| acid (DDA) for DDT | | | |
| 1-naphthol | Urine | | |
| (Carbory) | | | |
| Antibiotics | | | |
| Chloramphenicol | Milk, blood, urine | Anti- | Sigma |
| | | chloramphenicol | |
| Cephalexin (CEX) | Milk | | |

IASABC—Institute of Animal Science, Agricultural Biotechnology Center, Gödöllö, Hungary

TABLE 2
Evaluation results of six different solid phases
for the effective removal of matrix interferences.

| | | Matrix interference adsorption | | |
|---|---|---|---|---|
| | Brown | | Flow-through | |
| Type of | color | Interfering peak | Internal | Sample |
| solid phase | adsorption | adsorption (HPLC) | control | spot |
| SAX | Strong | No adsorption, peak | Present | None |
| | | appeared | | |
| PAS | Strong | adsorbed, peak | Present | None |
| | | disappeared | | |
| CN | None | No adsorption, peak | None | None |
| | | appeared | | |
| Octadecyl | None | No adsorption, peak | None | None |
| | | appeared | | |
| Diol | None | No adsorption, peak | None | None |
| | | appeared | | |
| AMINOPROPYL | Strong | adsorbed, peak | Present | Present |
| | | disappeared | | |

TABLE 3
Effect of NaHCO₃ concentration on the
adsorption of matrix interference peak by the
AMINOPROPYL solid phase material.

| | Interfering matrix peak area |
|---|---|
| Extraction Solution | (retention time at 10 min.) |
| 50% methanol/50% water | 963.5 peak area units (pau) |
| 50% methanol/1.5% aqueous NaHCO₃ | 479.45 pau |
| (1/1) | |
| 50% methanol/3% aqueous NaHCO₃ (1/1) | 396.25 pau |
| 50% methanol/4% aqueous NaHCO₃ (1/1) | 127.59 pau |
| 50% methanol/6% aqueous NaHCO₃ (1/1) | — |
| 50% methanol/8% aqueous NaHCO₃ (1/1) | — |

TABLE 4
Recoveries of OA by solid phase extraction using
AMINOPROPYL material for clean-up.

| OA concentration spiked into samples | Recovery (%) |
|---|---|
| (ng · g⁻¹) | (n = 5) |
| 2.5 | 81 ± 2 |
| 5 | 74 ± 1 |
| 10 | 84 ± 1 |
| 20 | 72 ± 1 |
| 40 | 74 ± 1 |

TABLE 5
Calculated partition coefficient values for the dispersion of
OA between the aminopropyl solid phase and the methanol/
5% aqueous NaHCO₃ (1/1, vol/vol) mobile phase.

| OA | | MeOH/5% | |
|---|---|---|---|
| Concentration | Aminopropyl | NaHCO₃ | Partition coefficient |
| (ng · g⁻¹) | (NH₂) | (1/1, vol/vol) | (K_{d} = solid/mobile) |
| 20 | 11.42 | 8.58 | 1.33 |
| 40 | 21.22 | 18.78 | 1.13 |
| 80 | 25.72 | 54.29 | 0.47 |
| 160 | 79.88 | 80.13 | 0.997 |

TABLE 6
Between day repeatabilities of the column-based
tandem solid-phase clean-up enzyme immunoassay
for roasted coffee spiked with OA standard.

| | Ochratoxin A concentration | | | | |
|---|---|---|---|---|---|
| | (ng · g⁻¹) | | | | |
| Day | 0 | 2 | 4 | 6 | 8 |
| 1 | −−− | −−− | +++ | +++ | +++ |
| 2 | −−− | −−+ | +++ | +++ | +++ |
| 3 | −−− | −−− | +++ | +++ | +++ |
| 4 | −−− | −−− | +++ | +++ | +++ |
| 5 | −−− | −−− | +++ | +++ | +++ |

−−− = intense blue (negative);
−−+ = less intense blue (slightly positive);
+++ = no color (very positive)

1-37. (canceled)

38. A device for detecting the presence of an analyte in an interfering fraction containing fluid or semi-fluid sample, said device comprising:
(a) a transparent housing,
(b) inlet means for the sample to be analyzed,
(c) outlet means, and
(d) at least two discrete superposed layers, being a first and a second layer through which at least a part of the sample is able to be transported in said order, in which said first layer comprises an adsorbent medium comprising a derivatized surface capable of actively adsorbing at least a part of the interfering fraction of the sample and in which said second layer comprises an adsorbent medium containing a receptor capable of specifically retaining the analyte.

39. Device according to claim 38 wherein said receptor is an antibody.

40. Device according to claim 38, in which said derivatized surface comprises at least one of the groups selected from octadecyl, octyl, ethyl, cyclohexyl, phenyl, aminopropyl, cyanopropyl, diol, n-propyl-ethylene-diamine, diethylaminopropyl trimethylaminopropyl, benzenesulfonylpropyl, sulfonylpropyl and carboxymethyl.

41. Device according to claim 38 consisting of two discrete superposed layers.

42. Device according to claim 38, in which said adsorbent medium is selected from the group consisting of agarose, silica, sepharose or dextrans.

43. Device according to claim 38, in which said inlet means are connectable to pressure means capable of exerting pressure upon said sample to force the transport of the sample from the inlet means to the outlet means.

44. Device according to claim 43, in which said housing is a syringe and in which a syringe plunger is the pressure means.

45. A method for detecting the presence of an analyte in an interfering fraction containing fluid or semi-fluid sample, said method comprising the following steps:
(a) applying the sample in a flow-through motion onto a two layer adsorbent medium in which the first layer is capable of actively adsorbing at least a part of the interfering fraction of said sample, and the second layer is capable of specifically retaining the analyte of interest in said sample, further characterized in that said adsorbent medium of the first layer comprises a derivatized surface,
(b) optionally washing the two layer adsorbent medium in order to remove possible color interference of the second layer,
(c) optionally applying a predetermined amount of a binder molecule onto said two layer adsorbent medium, said binder molecule capable of interacting with non-occupied analyte-receptor of the second layer,
(e) finally detecting the presence or absence of said analyte of interest.

46. The method according to claim 45, in which said analyte-receptor is an antibody specifically recognizing said analyte of interest in the sample under investigation.

47. Method according to claim 45 in which detecting the presence of the analyte of interest in the second layer is done visually or by suitable detector means.

48. The method according to claim 45, in which step (b) and (c) are present.

49. The method according to claim 45 comprising step (b) and step (c′) wherein in step (c′) a predetermined amount of a binder molecule, labeled with an enzyme or a bioluminescent, chemiluminescent, phosphorescent or fluorescent molecule, is applied onto said two layer adsorbent medium, said binder molecule capable of interacting with non-occupied analyte-receptor of the second layer.

50. The method according to claim 45, in which additional steps are present in between step (c) or (c′), and step (d) consisting of:
optionally washing said two layer adsorbent medium in order to remove all unbound binder molecule from the second layer, and
applying a substrate onto said two layer adsorbent medium, said substrate capable of reacting with the binder molecule bound onto the non-occupied analyte-receptor of the second layer and capable of generating a detectable signal.

51. The method according to claim 45 comprising step (b) and step (c″) wherein in step (c″) a predetermined amount of the analyte molecule to be detected, labeled with an enzyme or a bioluminescent, chemiluminescent, phosphorescent or fluorescent molecule, is applied onto said two layer adsorbent medium, said labeled analyte molecule capable of interacting with non-occupied analyte-receptor of the second layer, and able to provide detection of the absence or presence of the analyte of interest in the second layer,

52. The method according to claim 51 in which additional steps are present in between step (c″), and step (d) consisting of:
optionally washing said two layer adsorbent medium in order to remove all unbound labeled analyte molecule from the second layer, and
applying a substrate onto said two layer adsorbent medium, said substrate capable of reacting with the labeled analyte molecule bound onto the non-occupied analyte-receptor of the second layer and capable of generating a detectable signal.

53. The method according to claim 45, further comprising pre-treating the sample under investigation by dissolving or extracting it with a specific solvent prior to applying said sample onto the two layer adsorbent medium, wherein the pretreatment extracts, concentrates or dissolves the analyte from the sample.

54. The method according to claim 45, in which said sample is applied onto said two layer adsorbent medium by means of pressure.

55. The method according to claim 45, in which the analyte in said sample under investigation is a member selected from the group consisting of toxins, mycotoxins, pesticides, drugs, antibiotics, hormones, and their respective conjugates, metabolites and derivatives.

56. The method according to claim 55, in which the analyte in said sample under investigation is a mycotoxin.

57. The method according to claim 56, in which the analyte in said sample under investigation is ochratoxin A.

58. A kit consisting of a device according to claim 38, and one or more of the following:
(a) a pretreatment solvent capable of extracting, concentrating or dissolving the analyte of interest in the sample under investigation,
(b) pressure means connectable to the inlet means of the device and capable of forcefully exerting pressure upon said sample under investigation, to force at least part of said sample from the inlet to the outlet means of said device,
(c) a washing solution capable of removing possible color interferences of the second layer,
(d) a binder capable of interacting with non-occupied analyte-receptor of the second layer,
(e) a labelled binder molecule capable of interacting with non-occupied analyte-receptor of the second layer,
(f) a labelled derivative of the analyte molecule under investigation,
(g) a washing solution capable of removing all unbound binder or unbound labeled binder or unbound labeled analyte molecule from the second layer, and
(h) a substrate capable of reacting with said binder or labeled analyte molecule bound onto non-occupied analyte-receptor of the second layer, and capable of generating a detectable signal.

59. A solid phase cleaning up method for removing an interfering fraction from a fluid or semi-fluid sample, said method comprising applying the sample in a flow-through motion onto an adsorbent medium which is capable of actively adsorbing at least a part of the interfering fraction of said sample, characterized in that said adsorbent medium comprises a solid support material selected from the group consisting of agarose, silica, sepharose or dextrans and wherein at least part of the surface of said solid support material is derivatized to produce a bonded matrix.

60. A device operable in the method of claim 59 for removing an interfering fraction from a fluid or semi-fluid sample comprising an adsorbent medium which is capable of actively adsorbing at least a part of the interfering fraction of said sample, characterized in that said adsorbent medium comprises a solid support material selected from the group consisting of silica derivatives and wherein at least part of the surface of said solid support material is derivatized.

61. Device according to claim 60 in which said derivatized surface comprises at least one of the groups selected from octadecyl, octyl, ethyl, cyclohexyl, phenyl, aminopropyl, cyanopropyl, diol, n-propyl-ethylene-diamine, diethylaminopropyl, benzenesulfonylpropyl, sulfonylpropyl, carboxymethyl and trimethylaminopropyl.

62. Device according to claim 61 in which said absorbent medium is a bonded silica solid phase or an aminopropyl solid phase.

63. A device according to claim 38 further comprising in a predetermined space of said second layer a predetermined amount of the analyte molecule to be detected, labeled with an enzyme or a bioluminescent, chemiluminescent, phosphorescent or fluorescent molecule.
